(19) United States
(12) Patent Application Publication
(10) Pub. No.: US 2019/0374277 A1
(43) Pub. Date: Dec. 12, 2019

(54) Tissue Ablation System with Deployable Tines

(71) Applicants: Actuated Medical, Inc., Bellefonte, PA (US); The Penn State Research Foundation, University Park, PA (US)

(72) Inventors: Roger B. Bagwell, Bellefonte, PA (US); Kevin A. Snook, State College, PA (US); Bradley W. Hanks, State College, PA (US); Mary I. Frecker, State College, PA (US); Matthew T. Moyer, Hummelstown, PA (US); Charles Dye, Hershey, PA (US)

(73) Assignees: Actuated Medical, Inc., Bellefonte, PA (US); The Penn State Research Foundation, University Park, PA (US)

(21) Appl. No.: 16/437,971

(22) Filed: Jun. 11, 2019

Related U.S. Application Data
(60) Provisional application No. 62/683,085, filed on Jun. 11, 2018.

(51) Int. Cl. A61B 18/14

(52) U.S. Cl. CPC A61B 18/14; A61B 2018/00214

(57) ABSTRACT

A tissue ablation device includes an ablation stem insertable into the working channel of a medical device such as endoscope for radiofrequency ablation of target tissue. The stem includes a sheath, cannula, and ablation wire concentrically disposed within one another. Each of the sheath, cannula and ablation wire are independently controllable by a dedicated positioner located in the handset. An end effector of tines is removably attached to the distal end of the ablation wire and expand radially outwardly when deployed to form a three-dimensional ablation zone. A motor provides repetitive reciprocating vibrations to generate axial displacement of the ablation wire and tines for increased accuracy in insertion into tissue. RF ablation is also provided through the ablation wire and tines. The end effector tines are removable to remain in the target tissue as a fiducial marker for later procedures.

### CROSS-REFERENCE TO RELATED APPLICATIONS

The present application claims the benefit of U.S. Provisional Application Ser. No. 62/683,085, filed on Jun. 11, 2018, the contents of which are incorporated herein by reference in their entirety.

### STATEMENT REGARDING FEDERALLY SPONSORED RESEARCH OR DEVELOPMENT

This invention was made with government support under CA225169 awarded by the National Institutes of Health. The government has certain rights in the invention.

### FIELD OF THE INVENTION

The present invention generally pertains to minimally invasive medical and clinical research devices for the treatment of abnormal tissue such as cancer tumors. More specifically, the present invention pertains to a radiofrequency probe system that utilizes reciprocating motion to aid in penetration and advancement of electrodes through tissues within a body, and release of the electrodes for tracking or marking as a fiducial for subsequent secondary procedures such as but not limited to radiation therapy.

### BACKGROUND

Cancer is the second leading cause of death globally, and although improvements in treatment and technology have led to better prognoses overall, the asymptomatic nature of several early-stage cancers such as in the pancreas and liver still result in higher rates of morbidity and mortality compared to other types of cancer. As an example, although pancreatic cancer accounts for nearly 80% less diagnoses than breast cancer, it supplanted breast cancer as the third most deadly cancer in the U.S. in 2016, and worldwide, hepatocellular carcinoma is the third leading cause of cancer mortality.

Open surgical resection is the standard treatment for many cancer types because it provides the opportunity for the surgeon to directly visualize resection margins. However, the anatomic complexity surrounding the pancreas and liver limit this treatment option. Up to 70-80% of liver and pancreatic cancer patients are ineligible for resection surgery at diagnosis. Minimally invasive (MI) techniques are generally considered safer than open surgeries, and demonstrate lower levels of morbidity, mortality, and faster patient recovery. Though laparoscopy is a popular MI method for liver access, deeper tumors in the liver and pancreas remain difficult to access percutaneously due to intervening structures.

In the field of medicine or clinical research, minimally invasive devices for therapy or treatment of diseases such as cancer have gained significant momentum. In such devices, the need to introduce penetrating members into tissue may be necessary for reasons such as biopsy sample collection, application of radiofrequency energy for thermal ablation, placement of fiducials for marking of tumor location, or injection of medications or chemical therapies. These actions are exceedingly difficult in stiffer tissues or structures surrounded by very soft tissues, particularly in endoscopic procedures where the tortuous path of the endoscope significantly reduces any direct force transmittance between the proximal and distal ends of the endoscope.

Interventional endoscopic techniques and devices have advanced significantly. However, one continuing issue is that probes small enough to fit through the endoscope working channel may not adequately penetrate harder, solid tumors. Cases focused on fine needle aspiration with endoscopes have reported technical failures from the inability to penetrate hard lesions, insufficient force generation due to a combination of tumor type and location/orientation, and difficulty in correct positioning. Another shortcoming of needles is that as single electrodes, these generate ellipsoidal ablation patterns that may not match the shape of more spherical tumors. Radiofrequency ablation (RFA) is performed by inserting the needles into the tumor multiple tines from different angles, necessitating several intestinal or stomach wall punctures that increase the chance of damaging parenchyma through over-ablation.

Methods and devices using multiple electrode tines have been developed and refined to provide more spherical and uniform ablation patterns by using spring memory to deploy electrodes with a radially outward, arcuate configuration, such as in U.S. Pat. No. 6,050,992. However, these devices, such as in the case of the probe system in U.S. Pat. No. 6,050,992, are focused more on laparoscopy, detailing a straight cannula shaft with a total length of 5 cm to 30 cm, preferably from 10 cm to 20 cm. However, translation of such laparoscopic devices to endoscopy is not trivial. For instance, the longer path and multiple curves of the gastrointestinal tract navigated by endoscopic procedures may require and transmit longitudinal forces very differently in a suitable cannula of 110 cm to 170 cm in length rather than a much shorter needle or cannula as in U.S. Pat. No. 6,050,992 which does not have to contend with such length, twists and turns and dampening of torque and longitudinal forces between the handset and the target site.

In addition, ablation electrode must be insertable into tissue to reach the target tumor. They must therefore have a sufficient size and strength to pierce tissue. However, if used in an endoscopic procedure, the ablation electrode would have to be quite small and long to reach the site. Because of dampening of longitudinal forces over the distance and tortuous pathways required of endoscopic procedures, there would be too little force to insert the ablation electrode into tissue at that distance.

However, successful EUS-guided thermal ablation has a secondary benefit: several studies have shown that dual-energy therapies provide synergistic effects, including combination RFA and radiation therapy. Stereotactic body radiation therapy (SBRT) is a highly localized radiation therapy that uses multiple radiation beams directed from different angles to produce sharp dose gradients, thereby minimizing radiation to nearby healthy organs and tissues while highly dosing a tumor. The RFA appears to preferentially sensitize the fast multiplying tumor cells near the RFA margins for 24-48 hours by lowering their thermal threshold for coagulation, enabling SBRT to more effectively kill cells at the tumor margins that may have survived the RFA procedure.

One difficulty in SBRT is that the sharp focus necessitates precise positioning. Patients typically wear body contour masks to minimize motion and respiratory tracking is performed when pulsing the radiation generator. Tumors with low imaging contrast are difficult to target, and therefore multiple platinum or gold fiducial markers, or seeds, are frequently implanted to mark the three-dimensional structure of the tumors. Traditional fiducial seeds exhibit high contrast in computed tomography (CT) imaging, however they can migrate for several days following implantation, and a week delay is usually given before starting SBRT treatments to ensure multiple sessions do not treat different anatomical sites as the fiducials migrate. Although migration risks are generally low and acceptable migration is usually within 2 mm, approximately 2-6% migrate distances of 5 mm or more (up to several centimeters) and occasionally migrate out of the scanning area completely (gross migration).

A device with a structure that minimizes or eliminates migration within tissues would provide the capability to begin performing procedures such as SBRT within the ideal 24-48 hour window after ablation.

A need therefore still exists to improve the insertion of electrode probes by reducing the force required to insert them, perform adequate RFA of deep tumors, and place fiducial markers accurately and without significant migration from the tumor site. As such, there remains room for improvement within the art.

### SUMMARY

The present invention relates to a system that uses oscillation to deploy a tissue-penetrating electrode through an endoscope to treat tumors and enables implantation of the same electrode for dual function. Specifically, the present invention is directed to tissue ablation device for use as an accessory probe system deployable within the working channel of a medical device such as an endoscope, and which produces axially-directed oscillatory motion (also referred to as reciprocating motion) of an RFA electrode with a plurality of tines at the distal end for penetration and insertion of the RFA electrodes or tines into target tissue for RFA ablation. The RFA electrodes or tines are also releasable for remaining in the target tissue as a fiducial marker for subsequent treatment, such as with radiation therapy. Although described here generally as a medical device or endoscope, the tissue ablation device of the present invention may be used in the working channels of a wide variety of medical devices, such as but not limited to a gastroscope, duodenoscope, colonoscope, laparoscope, and pediatric versions of these.

The tissue ablation device comprises an ablation stem which is insertable into the working channel of a medical device for minimally invasive procedure. The ablation stem includes an outer sheath, a cannula disposed concentrically therein, and an ablation wire disposed concentrically therein. Each of the sheath, cannula and ablation wire may be telescopically disposed relative to the other stem components, and are each independently and selectively movable relative to the other stem components by its own dedicated positioner. The tissue ablation device also includes a handset that remains exterior to the medical device, such as endoscope, and is operated by a user to adjust each of the sheath, cannula and ablation wire positioners as desired for insertion, ablation and removal through the working channel of the medical device.

A plurality of tines or RFA electrodes are attached to the distal end of the ablation wire. When the ablation wire is retracted for navigation to the target site, the tines may be axially aligned with the ablation wire. When the ablation wire is deployed beyond the remainder of the stem, the tines extend radially outwardly from ablation wire, creating a three-dimensional zone for ablation, such as spherical, ellipsoid or otherwise shaped according to the length and curvature of the tines. The tines may also be separated from the ablation wire and left in the target tissue following ablation to act as a fiducial for follow-on activities.

The handset includes a motor as part of a displacement assembly configured to generate repetitive reciprocating or oscillatory vibrations that result in small displacements of the ablation wire, thereby reducing the forced required to penetrate through tissues. Reciprocating motion of the ablation member facilitates less tissue displacement and drag, enabling, for example, easier access between soft, healthy tissue and harder, partially-necrosed tumor tissue. Specific applications of the invention include, but are not limited to, penetration of tumor tissues in the pancreas, liver, kidney, bladder, or parynchema for delivery of radiofrequency energy and placement of a fiducial for follow-on therapy.

Accordingly, the present tissue ablation device is capable of use through the working channel of an endoscope for endoscopic RF ablation. It is able to produce larger ablation zones with a single puncture due to the expanding structure of the tines, which reduces time for treatment and potential complications from multiple tissue perforations to access all of the relevant target tissue for full site ablation. The repetitive reciprocating vibrations coupled with the ablation wire and tines during insertion provide enhanced trajectory control to achieve a more accurately coregistration of the tines with the target tissue for ablation. Finally, the releasable aspect of the tines for residence within the target tissue once implanted allows for a more reliable fiducial marker that is subject to less migration, allowing follow-on therapies to be administered sooner and with greater confidence of being applied to the same location as was previously ablated.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended Figs. in which:

FIG. 1 is a perspective view of one embodiment of the tissue ablation device of the present invention.

FIG. 2A is a cross-sectional detail view of the distal end of the ablation stem, shown in a deployed arrangement.

FIG. 2B is a diagrammatic view of the tines of ablation stem shown detached from the ablation stem and implanted in target tissue.

FIG. 3 is a cross-sectional detail view of the distal end of the ablation stem of FIG. 2, shown in a stowed or retracted arrangement.

FIG. 4 is an isometric view of the sheath positioner of the tissue ablation device.

FIG. 5A is a cross-sectional view of the handset attached to an endoscope with the sheath positioner in a retracted position.

FIG. 5B is a cross-sectional view of the handset of FIG. 5A with the sheath positioner in a deployed position.

FIG. 6 is a cross-sectional view of the handset of the tissue ablation device.

FIG. 7A is a partial cut-away view of the handset, showing the ablation positioner in a retracted position.

FIG. 7B is a partial cut-away view of the handset of FIG. 7A, showing the ablation positioner in an extended position.

FIG. 8 is a partially exploded view of the ablation positioner handle, handset housing and motor housing.

FIG. 9A is a partial cut-away view of the handset, showing the needle in a retracted position.

FIG. 9B is a partial cut-away view of the handset, showing the needle in a deployed position.

FIG. 10 is a cross-sectional view of the needle positioner of the tissue ablation device.

FIG. 11A is a perspective view of the needle positioner, shown in a retracted and unlocked position.

FIG. 11B is a perspective view of the needle positioner of FIG. 11A, shown in a deployed and locked position.

FIG. 12 is an exploded view of the displacement assembly of the tissue ablation device.

FIG. 13A is a cross-sectional view of the displacement assembly of FIG. 12, showing the proximal or reverse stroke.

FIG. 13B is a cross-sectional view of the displacement assembly of FIG. 13A, shown in a distal or forward stroke.

Like reference numerals refer to like parts throughout the various views of the drawings.

### DETAILED DESCRIPTION

Reference will now be made in detail to embodiments of the invention, one or more examples of which are illustrated in the drawings. Each example is provided by way of explanation of the invention, and not meant as a limitation of the invention. For example, features illustrated or described as part of one embodiment can be used with another embodiment to yield still a third embodiment. It is intended that the present invention include these and other modifications and variations.

It is to be understood that the ranges mentioned herein include all ranges located within the prescribed range. As such, all ranges mentioned herein include all sub-ranges included in the mentioned ranges. For instance, a range from 100-200 also includes ranges from 110-150, 170-190, and 153-162. Further, all limits mentioned herein include all other limits included in the mentioned limits. For instance, a limit of up to 7 also includes a limit of up to 5, up to 3, and up to 4.5.

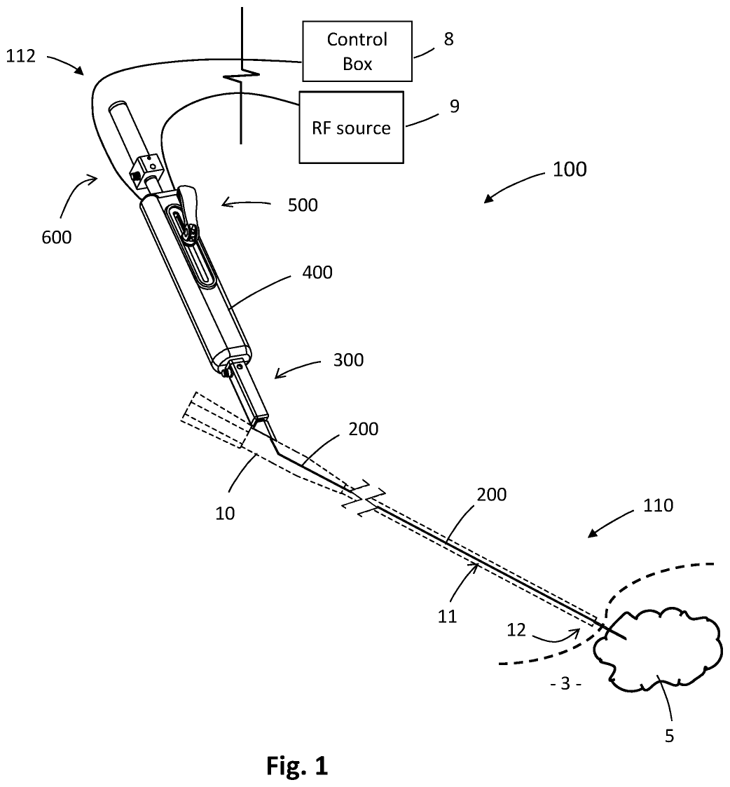

As shown in FIG. 1, the present invention is directed to a tissue ablation system 100 which is designed, to provide radiofrequency ablation to a target tissue 5, such as a tumor or cyst, which may be within a living being. For instance, the tissue ablation system 100 may be used with another medical device 10 for access into a living being, such as but not limited to an endoscope, laparoscope, or other invasive device for accessing the interior of an animal, which may be human, non-human primate, mammal, or non-mammalian animal. At least a portion of the tissue ablation device 100 may be dimensioned to fit within the working channel 11 of an endoscope. In at least one embodiment, this portion of the tissue ablation device 100 is the ablation stem 200, described in greater detail below. The ablation stem 200 is preferably located at a distal end 110 of the tissue ablation device 100 for entry into the medical device 10, and indeed may be connected to the medical device 10 such as an endoscope by a Luer® connector or other suitable selective connector. Accordingly, “distal” as used in this application refers to the end of the tissue ablation device 100 that is nearest the patient or target tissue 5 during use, such as for introduction into another medical device 10 for accessing the patient and/or tissue 3. Similarly, “distal” or “distally” may also refer to the direction of the medical device 10 and/or patient or tissue when in use. The tissue ablation device also includes a proximal end 112, which is opposite from the distal end 110. Accordingly, as used herein “proximal” or “proximally” refers to the end or direction away from the medical device 10 and/or patient or target tissue 5. The proximal end 112 may be held and operated by an operator or user of the tissue ablation device 100.

With reference to FIGS. 1, 2A and 3, the tissue ablation device 100 includes an ablation stem 200 at the proximal end 110. The ablation stem 200 is composed of a series of elongate members telescopically disposed one within another and which are collectively configured and dimensioned to fit within the working channel 11 of a medical device 10, such as but not limited to an endoscope or laparoscope. In some embodiments, the ablation stem 200 may be long in length, such as up to about 1.7 meters. In at least one embodiment, the ablation stem 200 may have a length in the range of about 100-200 cm, and in certain embodiments about 110-170 cm. Such length may be particularly useful in endoscopic treatments where the working channel is quite long since the entry point is often an existing orifice of the gastrointestinal system. The length and flexibility is needed to navigate the endoscope and ablation stem 200 inserted therein, from the entry point to the target tissue within the gastrointestinal system. In other endoscopic treatments, the endoscope may enter the patient through an artery, such as a femoral artery, and may be inserted a distance to the target tissue, such as the heart or lungs. In such embodiments, the ablation stem 200 is not only long but also sufficiently flexible to follow the endoscope in maneuvering through the gastrointestinal system or venous system without becoming kinked or twisted, which would reduce or prevent efficacy. In other embodiments, however, the ablation stem 200 may be shorter, such as up to 100 cm and in some embodiments more particularly may be in the range of about 5-30 cm or 10-20 cm. These shorter embodiments may be more useful in shorter distance applications such as laparoscopic procedures in which the medical device 10 and/or ablation stem 200 is inserted through an incision in the patient at or near the target tissue 5. In such embodiments, the ablation stem 200 need not be as long as may be required for endoscopic applications.

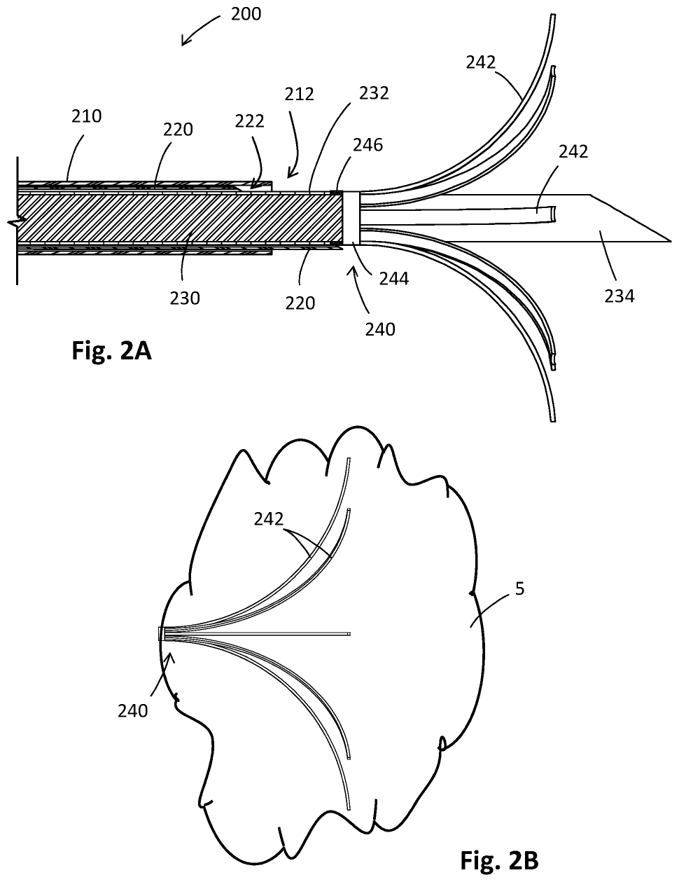

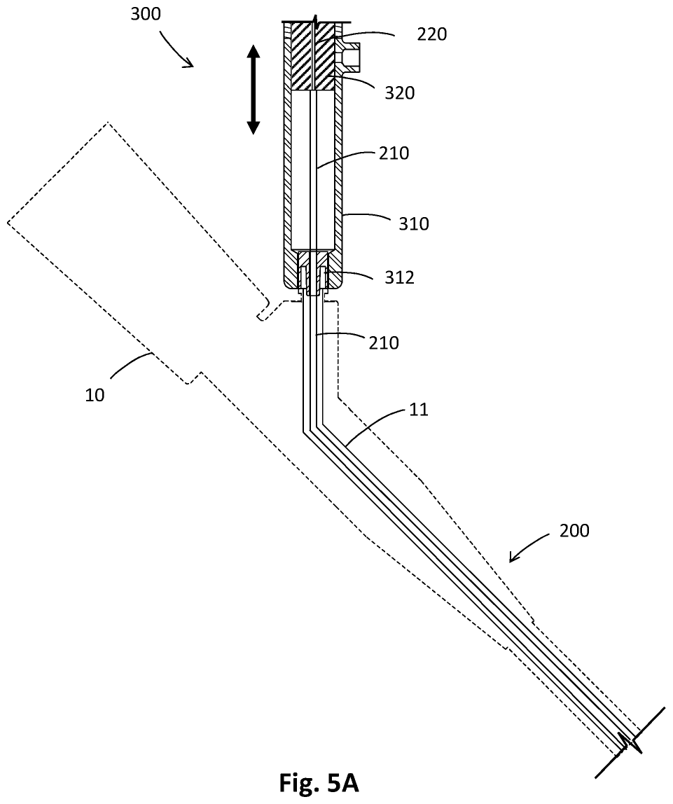

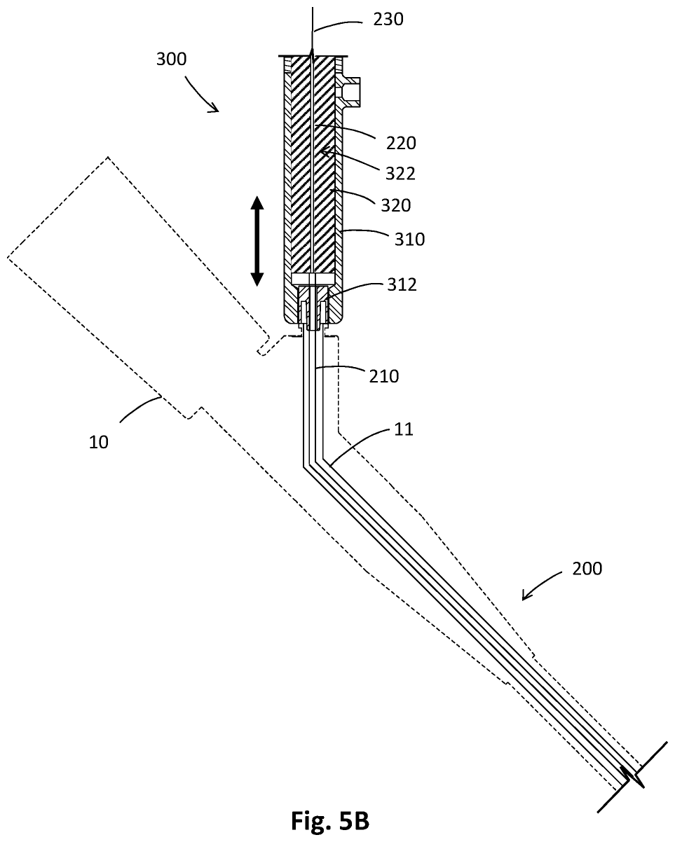

Regardless of the medical device 10 used, the ablation stem 200 includes a number of components concentrically disposed about one another and selectively moveable relative to one another, such as by telescopic movement. As best shown in FIG. 2A, the ablation stem 200 includes a sheath 210 that forms an outer surface of the ablation stem 200. The sheath 210 is hollow and surrounds and protects the remaining components of the ablation stem 200 during movement through the medical device 10 such as the working channel of an endoscope. Accordingly, the sheath 210 is made of material of sufficient durability to protect the other components within. Examples of suitable material include, but are not limited to Teflon®, nylon, polyimide and Pebax®. The sheath 210 may also include wire reinforcement for added durability while keeping the wall thin. The sheath 210 includes a sheath lumen 213 extending between an open distal end 212 and a proximal end where it attaches to a sheath extension member 320, as shown in FIGS. 4-5B discussed below. The sheath 210 has a diameter sized to fit within and be movable within a working channel 11 of a medical device 10 such as an endoscope. For instance, in some embodiments, the sheath 210 has an outer diameter of up to about 2.4 mm or about 15 Fr so it may fit within even small working channel 11 of current medical devices 10, which may measure about 2.8 mm in diameter. The inner diameter of the sheath lumen 213 may be up to about 1.8 mm.

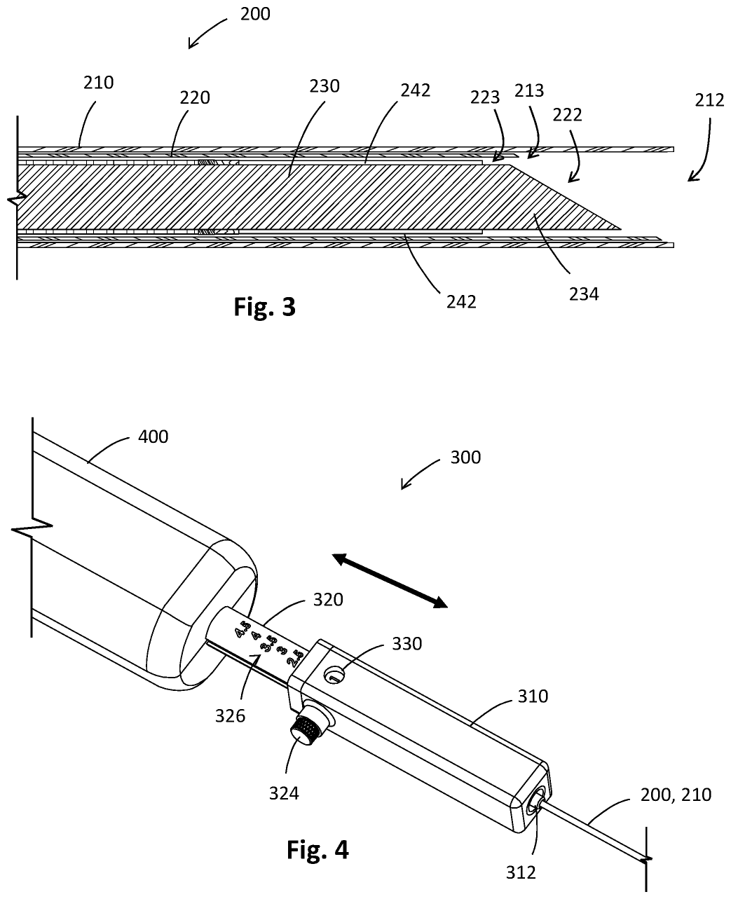

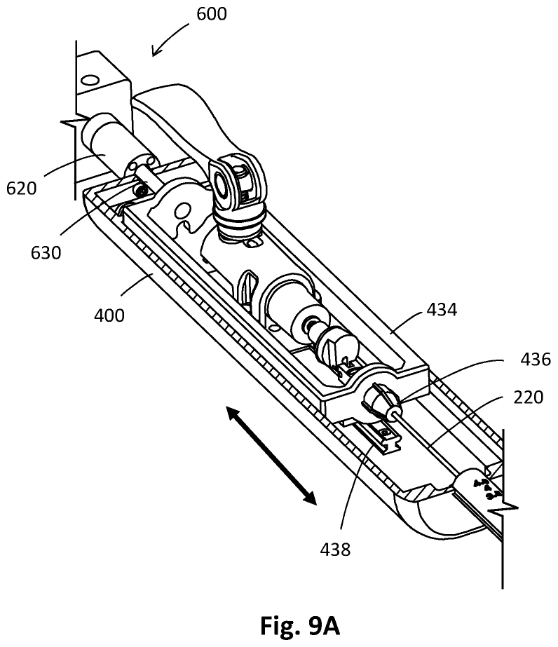

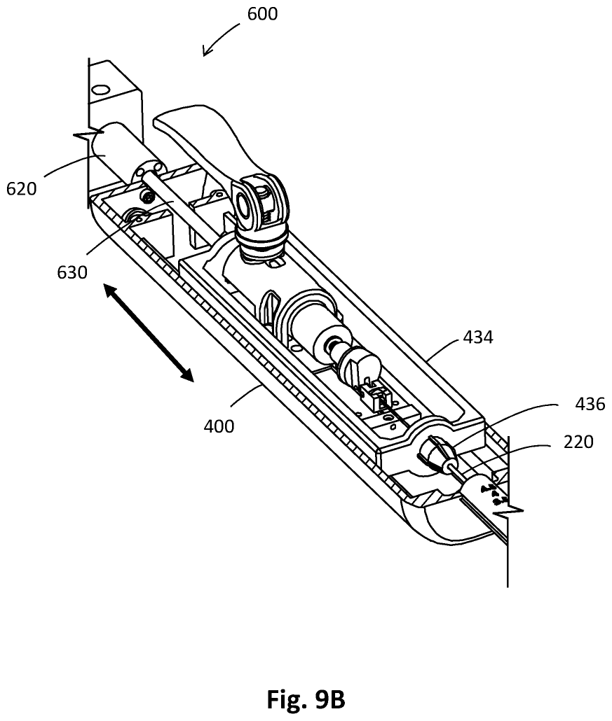

The ablation stem 200 also includes a cannula 220 disposed concentrically within the sheath 210. The cannula 220 is used to gain access to the general region of the target tissue 5. The cannula 220 may be any type of cannula, including but not limited to penetrating members such as needles, and may be made of materials which are preferably biocompatible such as but not limited to grade 316 stainless steel, Nitinol®, or titanium. The cannula 220 may be of any suitable size or gauge as will fit and be slidable within the sheath 210, such as 18-25 gauge but more preferably 19-22 gauge. The cannula 220 is also hollow with a lumen 223 extending therethrough and terminating in an open distal end 222, as shown in FIG. 2A. In at least one embodiment, the open distal end 222 of the cannula 220 may be sharp, such as tapered for easier insertion, although it is not required in all embodiments. The opposite proximal end of the cannula 220 is mounted to a second support member 434 within the handset 400, shown in FIGS. 9A-9B and described in greater detail below. The cannula 220 may be moved with the ablation stem 200 as a whole but is also selectively movable relative to the sheath 210 by engagement of a cannula positioner 600, discussed in greater detail below. The cannula 220 may be selectively moved through the sheath 210 in a distal direction through the open distal end 213 of the sheath 210 for deployment, as shown in FIG. 2A. When not deployed, such as for maneuvering through the medical device 10 working channel 11 to the target site, the cannula 220 remains fully within the sheath 210 in a retracted position, as shown in FIG. 3.

The ablation stem 200 further includes an ablation wire 230 disposed concentrically within the lumen 223 of the cannula 220, as depicted in FIGS. 2A and 3. The ablation wire 230 is made of a conductive material configured to transmit energy from an energy source connected at a proximal end to the distal end of the ablation wire 230. For instance, in a preferred embodiment the ablation wire 230 is configured to transmit radiofrequency (RF) energy from an RF source 9 to the distal end of the ablation wire 230 for ablation of target tissue 5. The ablation wire 230 may therefore be made of any suitable conductive material, such as but not limited to Nitinol® or titanium. The ablation wire 230 is sufficiently flexible to bend and flex with the navigation of the ablation stem 200 within the working channel 11 of a medical device 10 to reach a target tissue, even when distant from the insertion point. In some embodiments, the ablation wire 230 may be a corded, twisted or braided wire. In other embodiments, however, the ablation wire 230 may be smooth or otherwise without external texture. The ablation wire 230 may further be of any suitable size that fits within the surrounding cannula 220, and therefore has a smaller diameter than the cannula 220, such as but not limited to in the range of about 0.5-2 mm, and may be about 1 mm or 1.4 mm in certain embodiments. Additionally, though called a “wire,” it should be appreciated that in some embodiments the ablation wire 230 may be hollow and may transmit fluid therethrough to an opening at its distal end, such as for irrigating to provide liquids such as saline or medication to the target tissue, or to aspirate tissue and fluids such as blood, medication and saline from the target tissue, all of which may occur before, during or after target tissue ablation with RF energy.

Because the ablation wire 230 is configured to provide RF energy to the target tissue 5 for ablation, and because the surrounding cannula 220 is also conductive, in some embodiments the ablation wire 230 may be at least partially surrounded by insulating material 232, as best shown in FIG. 2A. The insulating material 232 may be any non-conductive material that would insulate the cannula 220 from the RF energy provided by the ablation wire 230. Examples of suitable material include, but are not limited to Teflon®, polyimide, polyvinylidene fluoride, polyurethane, polyolefin, polyvinyl chloride (PVC) and polyethylene. This keeps the RF energy from being transferred to the cannula 220, which would decrease the amount of RF energy delivered to the target tissue site for ablation and energize the cannula 220 with RF energy, possibly damaging the ablation stem 200. Both of these events are negative repercussions to be avoided which the insulating material 232 prevents. In some embodiments, the insulating material 232 is affixed to the ablation wire 230 and is movable therewith through the cannula 220. In other embodiments, the insulating material 232 may be disposed concentrically around the ablation wire 230 so that it may contact the ablation wire 230, but need not be affixed or secured thereto. In such cases, the insulating material 232 may form a sleeve around the ablation wire 230. In still other embodiments, the insulating material 232 may be affixed to the inner wall of the cannula 230.

The ablation stem 200 includes an end effector 240 at its distal end. The end effector 240 constitutes the distal-most portion of the ablation stem 200 that extends from the working channel of the medical device 10 and engages the target tissue 5 for ablation. As best shown in FIGS. 2A and 2B, the end effector 240 includes a distal tip 234 of the ablation wire 230 which is located at the distal end region of the ablation wire 230 and is configured to contact and/or pierce the target tissue. In at least one embodiment, the distal tip 234 of the ablation wire 230, and therefore of the end effector 240, may be sharp, such as angled, beveled or tapered for easier insertion into, piercing or penetration of the target tissue. In other embodiments, the distal tip 234 may be blunted, rounded or otherwise shaped for contacting the surface of the target tissue. In some embodiments, the distal tip 234 is simply the terminal end of a continuous ablation wire 230 and is made of the same material. In other embodiments, however, the distal tip 234 may be separately formed and secured to the ablation wire 230, such as by a collar, crimping, welding, adhesive, or other suitable fastener, as shown in FIG. 2A. In such embodiments, the distal tip 234 may be made of the same or different material as the ablation wire 230 but is nevertheless a conductive material for conveying RF energy from the ablation wire 230 to the target tissue.

The end effector 240 also includes a plurality of tines 242 removably connected to the distal tip 234 of the ablation wire 230. There may be any number of tines 242 present, such as but not limited to three, four, six, eight, nine, or twelve. The tines 242 are very fine, generally about 200-500 microns in diameter in order to pass through a working channel 11 of a medical device 10. In some embodiments there may be one central tine that is slightly larger than the remaining tines 242, such as 400 microns in diameter for the central tine compared to a diameter of about 250 microns for the remaining tines 242. The tines 242 may be arranged in any configuration relative to the ablation wire 230. For instance, in at least one embodiment the tines 242 may be collectively disposed concentrically about the distal end of the ablation wire 230. The tines 242 expand radially outwardly or spread out upon movement in the distal direction once outside of the confines of the surrounding cannula 220, as shown in FIG. 2A, so as to provide a generally three-dimensional ablation zone when RF energy is applied. The size and shape of the ablation zone depends on the length of the tines 242. For example, in at least one embodiment, the tines 242 may be up to about 3 cm, more preferably about 2-2.5 cm, and a spherical ablation zone is similarly sized. The resulting ablation zone may be any three-dimensional configuration as dictated by the length and shape of the tines 242. Examples include, but are not limited to, spherical, ellipsoid and irregular three-dimensional configurations.

In some embodiments, the tines 242 may be made of a flexible or resilient material and/or of a biasing configuration such that the tines 242 may have a spring force or bias force that permits them to be retained along the distal end of the ablation wire 230 when stowed in the ablation stem 200 for navigation through the medical device 10 to a target site, as shown in FIG. 3, and yet may automatically deflect or separate from the ablation wire 240 when the end effector 240 is advanced beyond the sheath opening 212 and/or the cannula opening 222, as shown in FIG. 2A. For instance, the tines 242 may be made of Nitinol®, spring steel or Inconel® material. The portion of the tines 242 not secured to the ablation wire 230 may expand radially outwardly from the ablation wire 230 once no longer restricted by the surrounding cannula 220 or sheath 210. As shown in FIG. 2A, the proximal ends of the tines 242 may be connected at a collar 244 to the ablation wire 230, but the opposite ends of the tines 242 and the remainder of their length may not be connected to anything. The free distal ends move away from the ablation wire 230 upon deployment of the end effector 240, increasing the distance between the distal ends of the tines 242 with the increased distance the end effector 240 is advanced from the ablation stem 200. Furthermore, each individual tine 242 may deflect a different distance, degree, or amount from the ablation wire 230 upon deployment of the end effector 240, but in at least one embodiment the tines 242 each independently deflect the same or similar distance from the ablation wire 230 to form a generally round or spherical pattern at the distal-most end of the end effector 240 when deployed. The spread of the tines 242 when deployed is inversely related to the insertion speed.

Each tine 242 may be connected to the ablation wire 240 directly, or in some embodiments the tines 242 may affixed to one another or a common collar 244 which at least partially encircles the ablation wire 230 at the distal tip 234. The tines 242 are selectively removably secured to the distal tip 234 of the ablation wire 230, either directly or at the tine collar 244, by a releasable fastener 246, shown in FIG. 2A. The releaseable fastener 246 may be any material that is can withstand reciprocating forces from repetitive displacement of the ablation wire 230 and transmit such reciprocating forces to the distal tip 234 and tines 242 without becoming dislodged or loosened, and yet can still be selectively removed when desired. Preferably, the fastener 246 is also biologically inert so as not to interfere with the target tissue. For example, the releaseable fastener 246 may be a biologically compatible polymer such as but not limited to polyethylene glycol (PEG) such as having molecular weights of 1,500-40,000 daltons, or more preferably 4,000-10,000 daltons, or compounds derived from materials such as alginate, polylysine, and gelatin, which may be dissolved by the application of a suitable corresponding solvent to the fastener 246. Accordingly, the fastener 246 may be chemically removed by the application of the appropriate solvent. For example, water could be used as the solvent to dissolve a fastener 246 formed of PEG, alginates, polylysine or gelatin. The solvent may be supplied to the fastener 246 by injection through the inner diameter of the cannula 220 from the proximal end to the distal end. Only enough solvent may be supplied as will dissolve the fastener 246 when so desired, so the solvent does not interfere with or negatively affect the target tissue or RF ablation at the site. Examples include but are not limited to 1 microliter of water to dissolve 600 micrograms of PEG or 1 microliter of an ethanol-water solution to dissolve 400 micrograms of PEG. In other embodiments, the fastener 246 may be physically dislodged from the ablation wire 240, such as by melting from the application of heat generated as the RF energy is supplied through the ablation wire 230 during ablation. For instance, the fastener 246 may be PEG 4000 which melts at temperatures of about 53° C. to 58° C., and RF energy of about 20 Watts provided for about 15 minutes time during ablation treatment generates temperatures in the range of 55° C. to 70° C., preferably about 60° C., though temperatures up to 100° C. are possible.

Separation of the end effector 240 from the ablation stem 200 may be desired when the distal end of the ablation stem 230 has reached the target tissue site and is deployed with the tines 242 implanted into the target tissue 5. Application of the appropriate separating mechanism, such as solvent for chemical removal or particular vibration for mechanical removal of the fastener 246 may be applied when the tines 242 are implanted at the desired location in the target tissue. In at least one embodiment, the tines 242 are preferably deployed and implanted into cancerous tissue within an organ, such as within a tumor or cyst in the gastrointestinal tract, pancreas, liver, heart, lung, kidney or other organ. Once the fastener 246 is removed, the tines 242 remain lodged or anchored within the target tissue, as shown in FIG. 2B. These tines 242 may then act as a fiducial for spatial tracking and subsequent therapy, such as but not limited to radiation or SBRT. Because the configuration of the tines 242 allow for a more three-dimensional implanting than other known fiducials, and particularly in embodiments in which the tines 242 are held together by a collar 244 or other shared structure, the tines 242 do not drift or migrate within the target tissue 5 once implanted as much as other single fiducials do. This allows subsequent radiation therapy to be applied much sooner than is currently done, such as 24 to 48 hours after implantation, as compared to the days or weeks needed currently for typical fiducials to stop migrating or drifting within target tissue before radiation treatment can begin. This greatly increases the speed with which treatment can be applied and may increase positive outcomes since ablation sensitizes the tumor and radiation applied sooner thereafter may have an increased effect due to this sensitization.

The proximal end of the ablation stem 200 connects to the proximal end of the handset 400 of the tissue ablation device 100, as shown in FIG. 1. Each of the sheath 210, cannula 220 and ablation wire 230 attach to different parts of the handset 400 and each has their own positioner 300, 600 and 500, respectively, for selective and independent movement of the respective component of the ablation stem 200. These positioners 300, 600 and 500 will now be described in greater detail.

The tissue ablation device 100 includes a sheath positioner 300 located at the proximal end of the handset 400. As shown in FIGS. 4-5B, the sheath positioner 300 includes a sheath positioner handle 310 disposed at least partially surrounding a sheath extension member 320. The sheath positioner handle 310 includes a hollow interior space which is configure to movably receive the sheath extension member 320 therein. The hollow interior space is also in fluid communication with a connector 312 at the distal end of the sheath positioner handle 310 where the tissue ablation device 100 attaches to a medical device 10, such as a port on an endoscope allowing access to the working channel as shown in FIGS. 5A and 5B. The connector 312 may be any suitable type of connector, such as but not limited to a Luer® type connector for selective attachment. The connector 312 permits connection of the sheath positioner handle 310 to the medical device 10 while also allowing free movement of the sheath 210 and other components of the ablation stem 200 therethrough.

The sheath extension member 320 is an elongate member extending from the proximal end of the handset 400 of the device 100. The sheath extension member 320 may be integrally formed with the handset 400 or may be attached to and extending from the handset 400. The sheath extension member 320 may also be hollow or have a channel 322 extending therethrough from the proximal end to the distal end.

The proximal end of the ablation stem 200 passes through the connector 312. The proximal end of the sheath 210 is secured to the sheath extension member 320, such as to a terminal end of the sheath extension member 320 or area proximate thereto, though it is contemplated the sheath 210 may be affixed to any location along or within the sheath extension member 320. The sheath 210 may be secured or affixed to the sheath extension member 320 by any suitable means, such as by bonding, adhesive, welding, or other permanent attachment. Regardless of attachment mechanism, the sheath 210 is attached to the sheath extension member 320 in such a way that does not affect or reduce the diameter of the sheath lumen 213. Accordingly, the cannula 220 and ablation wire 230 remain freely movable through the sheath lumen 213 at the point where the sheath 210 affixes to the sheath extension member 320. Indeed, as is clear from FIGS. 5A and 5B, the cannula 220, and ablation wire 230 disposed therein, continue proximally past the point where the sheath 210 attaches to the sheath extension member 320, through the channel 322 of the sheath extension member 320 and on to the handset 400 (not shown).

The sheath positioner handle 310 and sheath extension member 320 are selectively movable relative to one another, such as in the axial direction of the length of the sheath extension member 320. In at least one embodiment, the sheath positioner handle 310 is telescopically disposed over the sheath extension member 320 such that moving either the sheath positioner handle 310 or the sheath extension member 320 relative to the other either inserts or removes more of the sheath extension member 320 from the sheath positioner handle 310, depending on the direction of movement. For instance, in a retracted position of the sheath positioner 300 as seen in FIG. 5A, a minimal amount of the sheath extension member 320 is received within the sheath positioner handle 310. The sheath 210, being affixed to the sheath extension member 320, is therefore more proximally positioned within the working channel 11 of the medical device 10 and may indeed be fully retained with the working channel 11. In a deployed position of the sheath positioner 300, as in FIG. 5B, at least one of the sheath positioner handle 310 and sheath extension member 320 are moved relative to one another to increase the amount of sheath extension member 320 retained within the sheath positioner handle 310. Accordingly, the sheath positioner handle 310 and sheath extension member 320 are axially slidably adjustable relative to one another. This adjustment moves the sheath 210 axially distally so it extends through the open distal end 12 of the working channel 11 of the medical device 10 and beyond into the surrounding area, such as the gastrointestinal tract.

The sheath positioner 300 also includes a fastener 324 such as a thumbscrew, set screw or other suitable fastener that may be selectively tightened to secure the sheath positioner handle 310 and sheath extension member 320 together when the desired sheath 210 position is achieved. The fastener 324 is also configured to be selectively released to decouple the sheath positioner handle 310 and sheath extension member 320 when repositioning is desired. In at least one embodiment as in FIG. 4, the fastener 324 may extend through the sheath positioner handle 310 and may be selectively tightened onto the sheath extension member 320 to affix the two components together. In other embodiments, however, the fastener 324 may extend from the sheath extension member 320 to or through the sheath positioner handle 310.

The sheath positioner 300 may also include indicia 326 to facilitate accurate adjustment and placement of the sheath 210 relative to the working channel 11 of the medical device 10. In a preferred embodiment, the ablation stem 200 is inserted into the working channel 11 of an associated medical device 10 through the connector 312 discussed above. The length of the sheath 210 is adjusted to be approximately the same or similar length as the working channel 11. This allows the ablation stem 200 and its components to be located as close to the target tissue as possible for subsequent action. The indicia 326 may facilitate this locational accuracy. The indicia 326 may be numbers, lines, symbols, colors, patterns, shapes or other similar markings located along the sheath positioner 300 that provide an indication of the length of sheath 210 extending from the sheath extension member 320, or the distance from the distal end of the sheath 210 to the open distal end 12 of the working channel 11 of the medical device 10. The indicia 326 may be located anywhere on the sheath positioner 300. In some embodiments, as in FIG. 4, the indicia 326 may be located along the sheath extension member 320 for easy viewing to a user. The sheath positioner 300 may be secured with the fastener 324 based on the proximal edge of the sheath positioner handle 310 relative to the indicia 326 on the sheath extension member 320. In certain embodiments, the sheath positioner 300 may include a viewing aperture 330, such as may extend through the sheath positioner handle 310. The fastener 324 may be secured when the appropriate indicia 326 is viewable through the viewing aperture 330, indicating the desired alignment between the sheath positioner handle 310 and sheath extension member 320.

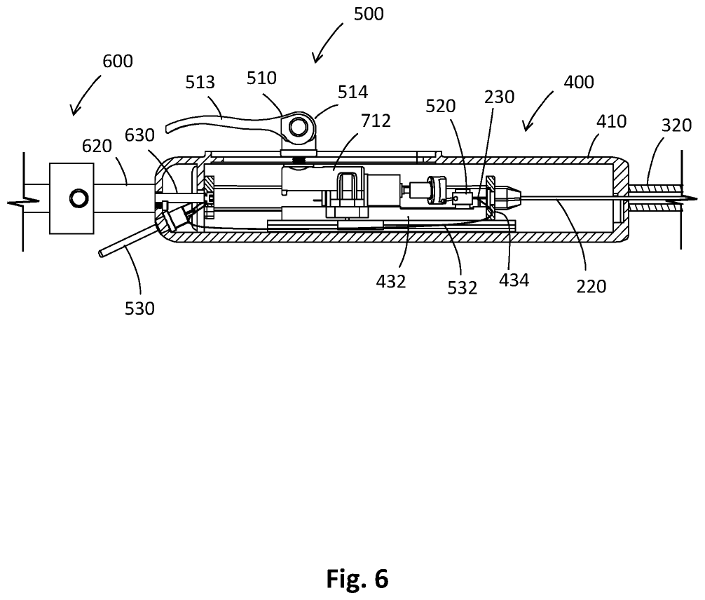

The tissue ablation device 100 also includes a handset 400 from which the sheath extension member 320 extends at the distal end. As shown in FIG. 6, the handset 400 includes a handset housing 410 which is the external structure defining an interior space of the handset 400 in which many of the internal components of the tissue ablation device 100 reside. The handset housing 410 may be held by a user when using the device 100. As mentioned previously, the sheath extension member 320 extends from the distal end of the handset 400. Specifically, the sheath extension member 320 may extend from the handset housing 410. In at least one embodiment the sheath extension member 320 is integrally formed with the handset housing 410. In other embodiments, however, the sheath extension member 320 may be securely affixed to the handset housing 410. The channel 322 of the sheath extension member 320 is in fluid communication with the interior space of the handset 400. Accordingly, the cannula 220 and ablation wire 230 disposed therein extend through the channel 322 of the sheath extension member 320 and into the interior space of the handset 400.

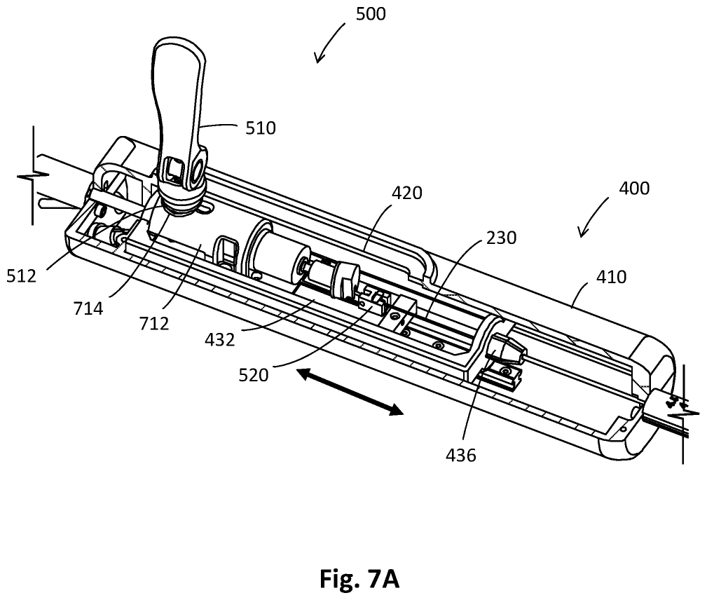

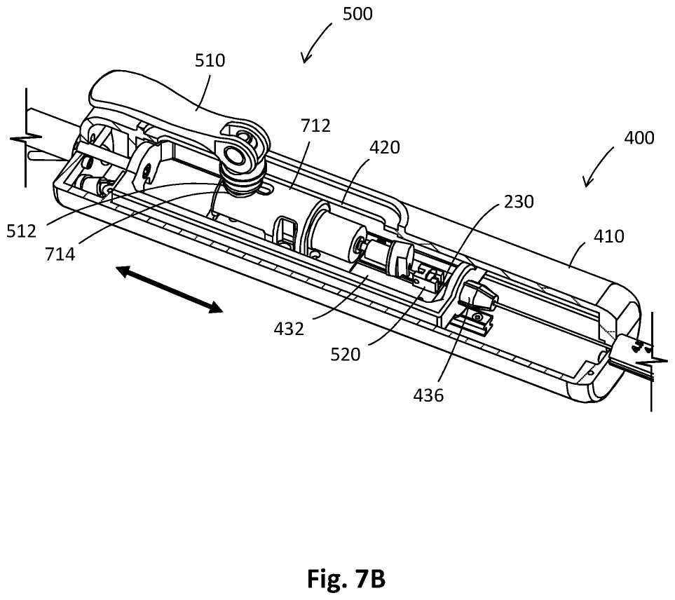

The handset 400 also includes a first support member 432 and second support member 434 each axially movable selectively and independently relative to one another and to the handset housing 410. In at least one embodiment as shown in FIGS. 7A-7B, the first support member 432 is slidably retained within the second support member 434 such that the second support member 434 prevents lateral movement of the first support member 432, limiting it to only axial movement along the longitudinal axis of the handset 400. The second support member 434 includes an attachment point 436 configured to receive and selectively retain the proximal end of the cannula 220. The attachment point 436 may include any suitable hardware, such as as Luer® type connector, a torquer, or other like connector. The proximal end of the cannula 220 connects to the attachment point 436, which may be tightened down to provide a secure connection to the cannula 220 but avoids compressing the inner lumen 223 of the cannula 220. Therefore, the ablation wire 230 remains freely movable through the cannula 220 at the attachment point 436. Accordingly, the cannula 220 is movable with the second support member 434.

The first support member 432 may be configured to retain an ablation wire mount 520, as shown in FIG. 6. The ablation wire mount 520 may be a single piece or may be multiple pieces collectively providing an anchor for the ablation wire 230. The proximal end of the ablation wire 230 is secured to or retained within the ablation wire mount 520. In turn, the ablation wire mount 520 may be secured to, either directly or indirectly, the first support member 432. Accordingly, the ablation wire 230 is movable with the first support member 432. In some embodiments, the ablation wire mount 520 may include a first part that is secured to the first support member 432 and a second part that is removable from the first part. The second part may receive the ablation wire 230 and may be removably insertable into and out of the first part of the ablation wire mount 520, such as in embodiments where the ablation wire 230 may be disposable but the handset 400 and its components may be reusable.

With reference to FIGS. 7A and 7B, the handset housing 410 includes a slot 420 extending therethrough along at least a portion of the length of the handset housing 410. The slot 420 is aligned with the longitudinal axis of the handset 400, and therefore of the first and second support members 432, 434 retained within. The slot 420 is dimensioned to receive and slidably retain a portion of the ablation positioner 500 therein for selective movement of the ablation positioner 500.

The ablation positioner 500 includes an ablation positioner handle 510 which is exterior to the handset 400 in at least one embodiment for selective actuation by a user to move the ablation wire 230 axially within the ablation stem 200. The handle 510 may include an elongate portion 513 which extends from a rounded pivot portion 514 about which the handle 510 may be moved. The pivot portion 514 may have an oblong shape resulting from an irregular radius, such that the radius is smaller along the axis of the elongate portion 513 and is larger in the direction perpendicular to the axis of the elongate portion 513. Accordingly, the pivot portion 514 is shorter in the unlocked first position shown in FIG. 7A and is longer in the locked second position shown in FIG. 7B.

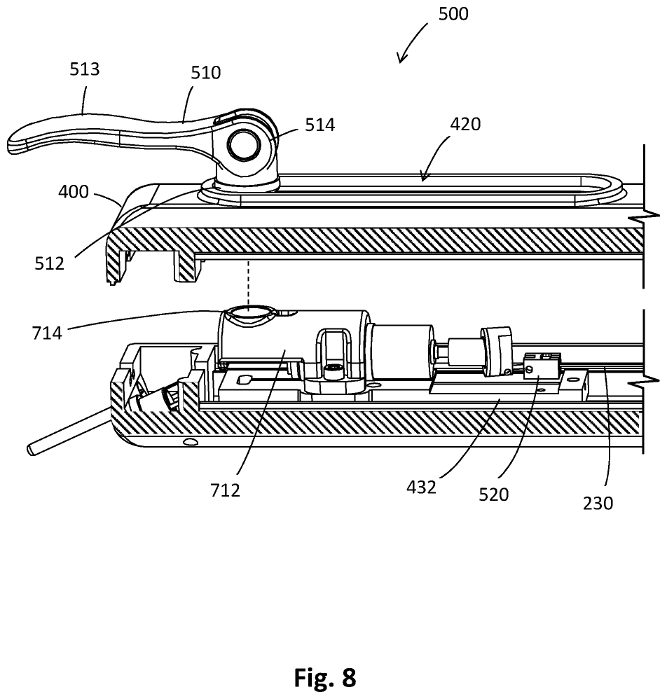

The ablation positioner 500 also may include a buffer 512 extending from the ablation positioner handle 510 and through the slot 420 of the handset housing 410. The buffer 512 may be made of a resilient or elastomeric material, such as but not limited to PVC, polyurethane or silicone, that may be compressed and return to its original shape when no longer compressed. Accordingly, the buffer 512 may act as a cushion between the ablation positioner handle 510 and the handset housing 410 when the ablation positioner handle 510 is in a locked position, as in FIG. 7B. The buffer 512 contacts the ablation positioner handle 510 on one side of an internal structure of the handset 400 connected to the first support member 432 on the other side. For instance, in at least one embodiment, the buffer 512 may be received within a seat 714 of a motor housing 712 within the handset 400, as shown in FIGS. 7A-8. The motor housing 712, in turn, may be affixed to the first support member 432. Accordingly, axial movement of the motor housing 712 moves the first support member 432 to which it is attached, which in turn moves the ablation wire mount 520 and the ablation wire 230. In other embodiments, the buffer 512 may contact and engage another component within the handset 400, including the ablation wire mount 520 in certain embodiments. In still further embodiments, there may not be a buffer 512, but the ablation positioner handle 510 itself may extend through the slot 420 in the handset housing 410 and directly engage a component within the housing 400 secured to the first support member 432 or ablation wire mount 520.

To move the ablation wire, the elongate portion 513 may be grasped by a user to move the ablation positioner handle 510 between a first unlocked position, as shown in FIG. 7A, and a second locked position such as shown in FIG. 7B. In the first unlocked position, the elongate portion 513 of the handle 510 extends away from the handset 400, such as perpendicular to or substantially perpendicular to the handset 400, though other angles are also contemplated in which the elongate portion 513 of the handle 510 is not parallel to the handset 400. In the unlocked position, the ablation positioner 500 may be moved axially along the slot 420 by applying force to the ablation positioner handle 510 in the axial direction, either distally to extend the ablation wire 230 or proximally to retract the ablation wire 230. The force is transferred to the first support member 432 through the contacting buffer 512 and motor housing 712, causing the first support member 432 to move axially with the force applied to the ablation positioner handle 510. Accordingly, the first support member 432 may be axially slidable relative to the handset housing 410 based on the force applied to the ablation positioner handle 510. It also moves independently from the second support member 434, such that force applied to the ablation positioner handle 510 does not affect the positioning of the second support member 434.

When the desired position is achieved, the ablation positioner 500 may be fixed in place by locking the ablation positioner handle 510, as in FIG. 7B. To lock the ablation positioner handle 510, force is applied to rotate the handle 510 about the pivot portion 514 until the elongate portion 513 is axially aligned with or parallel to the handset 400. As this occurs, the longer radius of the pivot portion 514 of the handle 510 presses onto the buffer 512, applying compressive force to hold the buffer 512 against the slot 420 through which it extends.

The ablation positioner 500 may be locked at any location along its axial movement when the desired position of the ablation wire 230 is achieved. For example, FIG. 7A shows the ablation positioner 500 in a retracted position in which the first support member 432 is proximally located withing the handset 400. The ablation wire 230 is retracted within the ablation stem 200 in this position. FIG. 7B shows the ablation positioner 500 in a deployed position in which the first support member 432 is distally located within the handset 400. The ablation wire 230 is deployed in this position to extend through the cannula opening 222 at the distal end of the ablation stem 200.

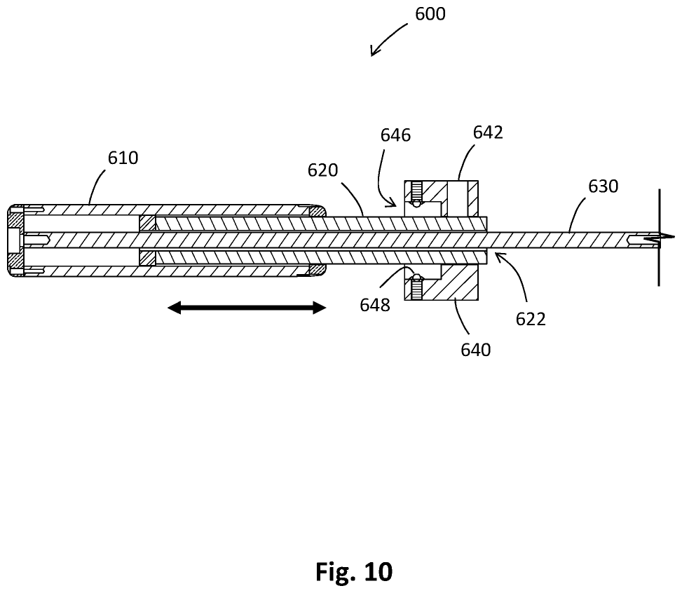

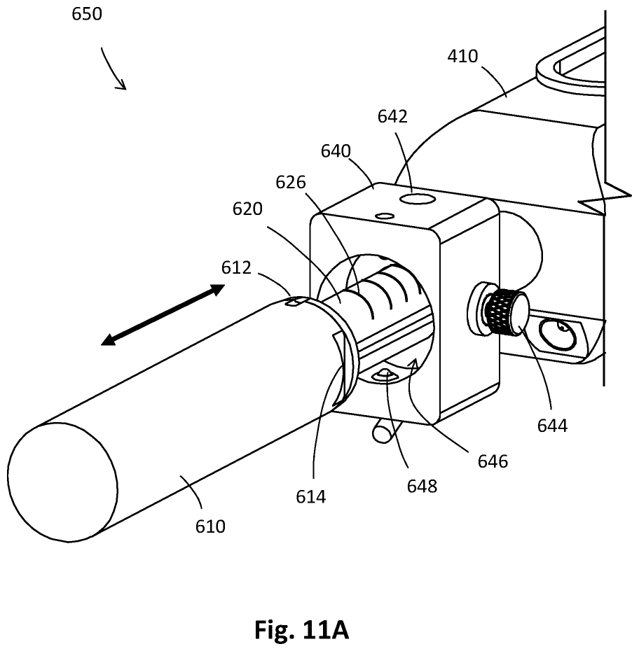

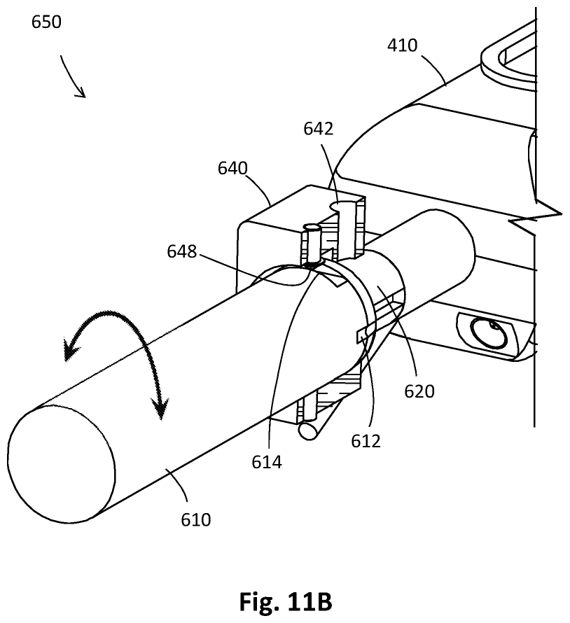

The tissue ablation device 100 also includes a cannula positioner 600, such as at the proximal end 112 as shown in FIGS. 9A-10. The cannula positioner 600 includes the cannula 220, the second support member 434 and the attachment point 436 at the distal end of the second support member 434 where the cannula 220 connects to the second support member 434 inside the handset 400. The cannula positioner 600 may also include a cannula extension member 620 which extends from the proximal end of the handset housing 410, as shown in FIGS. 9A-9B and 11A-11B. The cannula extension member 620 may be integrally formed with the handset housing 410 or may be secured to the handset housing 410. The cannula extension member 620 includes a channel 622 extending through its length, which is in fluid communication with the interior space of the handset 400.

The cannula positioner 600 also includes a cannula positioner handle 610 disposed at least partially around the cannula extension member 620. The cannula positioner handle 610 is configured to receive at least a portion of the cannula extension member 620 therein. In at least one embodiment, the cannula positioner handle 610 telescopically receives at least a portion of the cannula extension member 620 such that movement of the cannula positioner handle 610 either inserts or reveals more of the cannula extension member 620, depending on the direction of movement. The cannula positioner handle 610 is selectively movable relative to the cannula extension member 620 in an axial direction, such as slidably relative thereto. In at least one embodiment, axial movement of the cannula positioner handle 610 in the proximal direction relative to the cannula extension member 620 reveals more of the cannula extension member 620, whereas axial movement in the distal direction inserts more of the cannula extension member 620 into the cannula positioner handle 610. In at least one embodiment, the cannula extension member 620 may also include indicia 626, such as but not limited to numbers, lines, symbols, colors, patterns, shapes or other similar markings located along the length of the cannula extension member 620 that provide an indication of the length of cannula extension member 620 extending from the cannula positioner handle 610, which in turn is an indication of the length of cannula 220 extending from the handset 400 and through the ablation stem 200.

The cannula positioner 600 also includes a positioner shaft 630 which is secured to the cannula positioner handle 610 at one end, extends through the channel 622 of the cannula extension member 620, and is secured to the second support member 434 inside the handset 400 at its other end. In at least one embodiment, the positioner shaft 630 may be affixed to the proximal end of the second support member 434, though it may be secured to any location along the second support member 434. The positioner shaft 630 is of rigid construction such that movement of the cannula positioner handle 610 in turn moves the positioner shaft 630, which in turn moves the second support member 434. Accordingly, axial movement of the cannula positioner handle 610 will result in similar axial movement of the second support member 434, and therefore of the cannula 220 connected to the distal end of the second support member 434. The handset 400 may also include a track 436 along which the second support member 434 moves when the cannula positioner handle 610 is moved. Because the track 436 is axially disposed within the handset 400, the movement of the second support member 434 second support member 434 is therefore also axial and may be restricted to the length of the track 436 or the interior space of the handset 400.

The cannula positioner 600 is selectively movable between a retracted position, shown in FIG. 9A, and a deployed position, shown in FIG. 9B. In the retracted positioned, the second support member 434 is proximally disposed within the handset 400, retaining more of the cannula 220 within the handset 400 and ablation stem 200. To achieve this, the cannula positioner handle 610 is pulled proximally to extend the length of cannula extension member 620 exposed therefrom. When deployment of the cannula 220 is desired, the cannula positioner handle 610 is pushed distally, overlapping increasing length of the cannula extension member 620 and simultaneously moving the positioner shaft 630 and attached second support member 434 distally. The second support member 434 moves axially in the distal direction along the track 436 until movement is stopped, as seen in FIG. 9B. The cannula 220 attached thereto also moves distally within the ablation stem 200 and through the open distal end 212 of the sheath 210.

In certain embodiments, such as in FIGS. 10-11B, the cannula positioner 600 may include a fastener 644 to selectively secure the cannula positioner handle 610 to the cannula extension member 620 when the desired position is achieved. The fastener 644 may be any structure suitable for selective attachment, such as but not limited to a thumbscrew, set screw, bolt and wingnut, or other similar structure. In at least one embodiment, the fastener 644 may be on or extending through include a collar 640, as in FIG. 10, associated with the cannula extension member 620. For instance, the collar 640 may at least partially surround or encircle the cannula extension member 620 and is slidably movable therealong. The collar 640 may include a viewing aperture 642 extending therethrough so the indicia 626 on the cannula extension member 620 may be viewed through the collar 640 to assist in positioning of the collar 640. When the desired position of the collar 640 is achieved, the fastener 644 may be tightened to secure the collar 640 in place relative to the cannula extension member 620. In some embodiments, however, the fastener 644 may be located on or extend through the cannula positioner handle 610 and may be tightened when a desired position of the cannula positioner handle 610 is achieved to secure the cannula positioner handle 610 to the cannula extension member 620.

The collar 640 may also include a recess 646 formed therein, such as in an end which faces the cannula positioner handle 610, as shown in FIGS. 10-11B. The recess 646 is configured and dimensioned to receive at least a portion of the cannula positioner handle 610 therein. The collar 640 positioning is set and affixed to the cannula extension member 620 with a fastener 644. The cannula positioner handle 610 may then be moved distally relative to the cannula extension member 620 until a portion of it is received within the recess 646 of the collar 640, which stops its distal progression. The collar 640 may therefore be a depth control collar that prevents further axial movement of the cannula positioner handle 610 in one direction, such as distally. This controls the amount of cannula 220 that may extend beyond the working channel 11 of the medical device 12.

The cannula positioner 600 may also include a locking mechanism 650 to retain the cannula positioner handle 610, and therefore cannula 220, in a particular position once set. For instance, as shown in FIGS. 11A-11B, the locking mechanism 650 may include at least one groove formed in the cannula positioner handle 610. In at least one embodiment, the cannula positioner handle 610 may include at least one axial groove 612 formed along the longitudinal axis of the cannula positioner handle 610 and extending from the distal edge of the cannula positioner handle 610. The cannula positioner handle 610 may also include at least one circumferential groove 614 formed along at least a portion of the circumference of the cannula positioner handle 610. Accordingly, the circumferential groove(s) 614 runs perpendicular to the axial groove(s) 612. In some embodiments, the circumferential groove(s) 614 and axial groove(s) 612 do not intersect and are separate from one another. In other embodiments, however, the circumferential groove(s) 614 and axial groove(s) 612 may intersect and share a common groove recess. Regardless of whether separate or intersecting, the circumferential groove(s) 614 is aligned with at least a portion of the axial groove(s) 612. In at least one embodiment, the circumferential groove(s) 614 aligns with, such as is formed at the same distance from the edge of the cannula positioner handle 610 as the terminal end of the axial groove(s) 612, as shown in FIGS. 11A-11B.

The locking mechanism 650 also includes at least one protrusion 648 extending from the collar 640 into the recess 646. The protrusion(s) 648 may be made of a firm yet flexible material, such as a resilient plastic or polymer. The protrusions 648 may also be made of a spring-like or biasing material or construction, such as a spring plunger or similar structure that provides temporary deflection under pressure and resumes its shape once the pressure is no longer applied. Each protrusion 648 extends from the collar 640 by a length substantially equivalent to the depth of the axial and circumferential grooves 612, 614, and may have an overall shape or width substantially similar to the width of the axial and circumferential grooves 612, 614. As shown in FIG. 11A, there may be a similar number of protrusions 648 as there are at least axial grooves 612 in the cannula positioner handle 610. The axial grooves 612 and circumferential grooves 614 are configured to receive a protrusion 648 therein. For instance, each axial groove 612 is configured to receive a protrusion 648 as the cannula positioner handle 610 is advanced distally into the recess 646. The protrusion 648 is moved along the axial groove 612 as cannula positioner handle 610 is moved further distally. When the cannula positioner handle 610 is fully retained within the recess 646 and can go no further, the protrusion 648 may also have reached the terminal end of the axial groove 612. At this point, and with reference to FIG. 11B, the cannula positioner handle 610 may be rotated about the positioner shaft 630 with sufficient force to displace the protrusion 648 from the axial groove 612. The cannula positioner handle 610 continues to rotate under the protrusion 648 until the protrusion 648 reaches and is received within a circumferential groove 614. The circumferential groove 614 is sufficiently deep enough that it retains the protrusion 648 in the groove 614 and prevents axial movement. Therefore, the protrusion 648 within the circumferential groove 614 prevents axial movement of the cannula positioner handle 610, thereby locking the cannula positioner 600 in place.

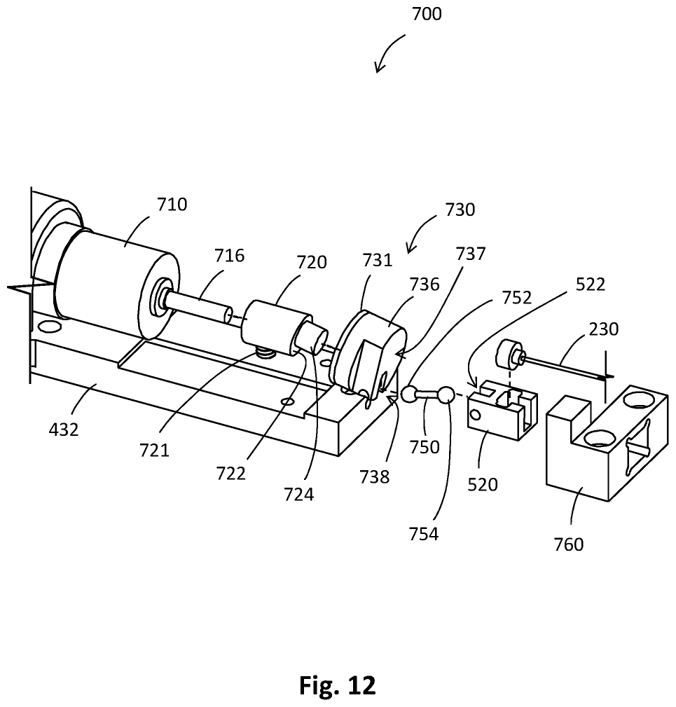

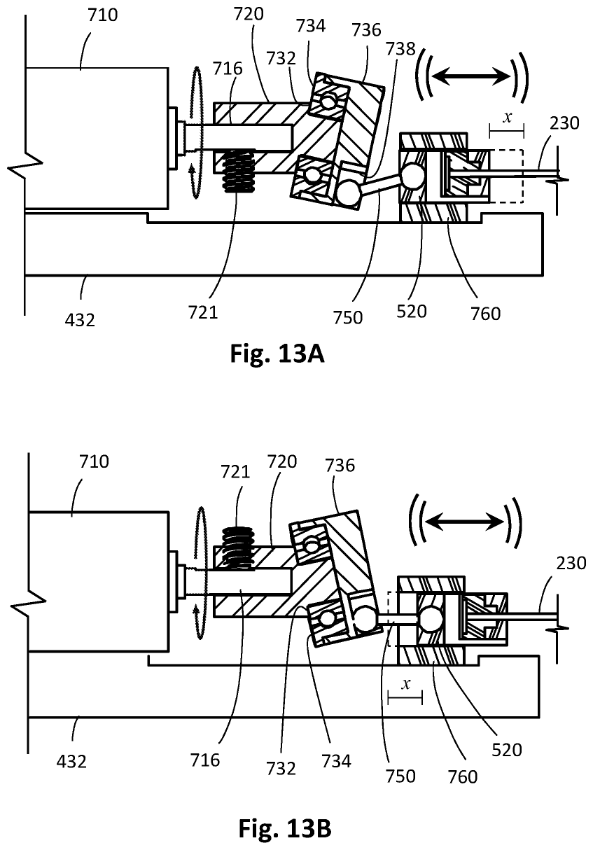

The tissue ablation system 100 also includes a displacement assembly 700 within the handset 400. The displacement assembly 700 is configured to generate and transmit axial vibrations to the ablation wire 230 and attached tines 242. FIG. 12 shows an exploded view of the components of the displacement assembly 700, and FIGS. 13A and 13B show the displacement assembly 700 in action. Specifically, the displacement assembly 700 includes a motor 710 retained within a motor housing 712. This motor 710 may be any type of motor, such as may be electrically activated. In at least one embodiment, the motor 710 may be a rotational motor such as a direct current (DC) or alternating current (AC) motor that is configured to provide rotational motion in the range of about 5-200 Hz and preferably in the range of about 10-40 Hz, such as about 35-40 Hz. These are just exemplary ranges, and higher frequencies are also contemplated, such as when using a piezoelectric actuator. These low frequencies are obtainable when operating a DC motor at about 3 volts. The motor 710 may be operated at more or less than 3 volts, such as up to about 6 volts, depending on the amount of torque desired. In other embodiments, however, the motor 710 may be configured to generate linear motion, such as voice coil motor (VCM), solenoid or piezoelectric actuators. In at least one embodiment, the motor 710 is configured to receive electrical signals from a control box 8, as shown in FIG. 1, which is in electrical communication with the motor 710 through a connection in the handset housing 410. The control box 8 may include switches, buttons, or other electrical components to turn the motor 710 on and off and to adjust the power, frequency, impedence, amplitude or other functional aspects of the motor 710. In other embodiments, the handset 400 may also include an on/off switch accessible at the handset housing 410 to turn the motor 710 on and off for selective generation of repetitive motion.

The motor 710 is retained within a motor housing 712, which may also include a seat 712 discussed above which interfaces with the buffer 512 of the ablation positioner 500. The motor housing 712 is secured to the first support member 432, as stated previously. The motor 710 includes a motor shaft 716 extending therefrom and through the motor housing 712, such as through the distal end as shown in FIG. 12. The motor shaft 716 is moved by the motor 710 when activated, such as in a rotational or axial direction. In the embodiment shown in FIGS. 12-13B, the motor 710 is a rotational motor, providing rotational motion to the motor shaft 716 such that the motor shaft 716 rotates 360° about its longitudinal axis.

The displacement assembly 700 further includes an adaptor 720 which is affixed to the motor shaft 716 opposite from the motor 710. Accordingly, the motor shaft 716 extends between the motor 710 and the adaptor 720. In at least one embodiment, as shown in FIGS. 12-13B, the adaptor 720 is configured to receive at least a portion of the length of the motor shaft 716 therein. A fastener 721 such as a thumbscrew, set screw or other similar structure may secure the adaptor 720 and motor shaft 716. In other embodiments, the motor shaft 716 and adaptor 720 may be affixed to one another, such as with bonding, welding, adhesive or other similar mechanism. Regardless of method of affixing, because the motor shaft 716 and adaptor 720 are secured to one another, the adaptor 720 rotates 360° with the motor shaft 716.

In the embodiment of FIGS. 12-13B, the adaptor 720 includes an angled face 722 and extension 724 extending therefrom opposite from the motor shaft 716. The angled face 722 is a planar surface that is off-axis from the axis of the motor shaft 716 and thus the portion of the adaptor 720 that interfaces with the motor shaft 716. The angled face 722 of the adaptor 720 is therefore included to change the angle from which the extension 724 proceeds from the adaptor 720, such that the extension 724 protrudes from the adaptor 720 in an axial direction, but one which is off-axis from or misaligned from the axis of the motor shaft 716. The degree of the angle of the angled face 722, and therefore of the extension 724 with respect to the axis of the motor shaft 716 alters the amount of displacement of the ablation wire 230 achieved by the displacement assembly 700. Accordingly, the adaptor 720 allows for greater axial displacements within a more compact footprint within the handset 400, as will be apparent from the full discussion of the displacement assembly 700. For instance, if the angled face 722 was perpendicular to the motor shaft 716, the extension 724 of the adaptor 720 would be concentric with the axis of the motor shaft 716 and no displacement of the ablation wire 230 would be achieved. The greater the angle of the angled face 722, the greater the angle deviation of the extension 724 relative to the motor shaft 716, and the greater the resulting displacement of the ablation wire 230. For instance, in at least one embodiment the angled face 722, and therefore the extension 724 protruding therefrom, has an angle in the range of 5 degrees to 30 degrees relative to the axis of the motor shaft 716, which results in a displacement in the range of 0.05 mm to 3 mm. In a preferred embodiment, the angle of the angled face 722 and extension 724 relative to the motor shaft 716 may be about 15 degrees to 20 degrees, providing a displacement of about 1.0 mm to 2.0 mm in the ablation wire 230.

The displacement assembly 700 may further include a bearing 730 having a plate 731 and a body 736. The bearing plate 731 is comprised of an inner ring 732 and a surrounding outer ring 734, where the inner ring 732 has a smaller overall diameter than the concentric outer ring 734. The inner diameter of the inner ring 732 is dimensioned to receive the extension 724 of the adaptor 720. The extension 724 of the adaptor 720 is affixed to the inner ring 732 of the bearing 730, such as by bonding, adhesive, welding or other like mode of attachment. Accordingly, the inner ring 732 of the bearing plate 731 rotates 360° with the adaptor 720.

The bearing body 736 is affixed to the outer ring 734 of the plate 731, such as by bonding, adhesive, welding or other suitable mode of secure attachment. As is common for bearing plates 731, the inner ring 732 and outer ring 734 are independently movable relative to one another by virtue of bearing balls gliding along the interface between the inner and outer rings 732, 734 which decouples rotational movement between the two rings. Accordingly, rotational movement of the inner ring 732 may move the interfacing balls within the bearing plate 731, causing some motion in the outer ring 734 but it does not transfer the rotational motion to the outer ring 734. Rather, the outer ring 734 is free to move as it will regardless of the rotation of the inner ring 732.

Because the inner ring 732 is affixed to the extension 724 of the adaptor 720, which is off-axis from the motor shaft 716, as the adaptor 720 rotates the extension 724 will follow a circular pathway. The diameter of the circular pathway will depend on the angle of the extension 724 and the degree of deviation from the axis of the motor shaft 716. For instance, larger angles of the angled face 722 and extension 724 result in larger diameters to the circular pathway followed by the distal end of the extension 724. The bearing 730 is affixed to the extension 724 and will therefore similarly be moved along the same circular pathway. This results in the body 736 of the bearing 730 following a circular motion, similar to the way a person's face moves as they angularly roll their head about on their neck. Because the outer ring 734 is freely movable along the interfacing balls of the bearing plate 731, the bearing body 736 does not rotate, but rather swings or rocks back and forth, such as up to 20°-40° in each direction as the distal face 737 of the body 736 follows an angular circular path. Accordingly, the bearing 730 may be considered a swash plate. As the distal face 737 follows this angular circular path, the top and bottom of the body 736 may be alternately more distally projecting. To be clear, the “bottom” of the body 736 is closer to the floor of the handset 400 and the “top” is closer to the side of the handset 400 having the slot 420. For instance, in a first position of the bearing 730 as shown in FIG. 13A, the top of the body 736 is more distally located compared to the bottom, such that the distal face 737 is downward facing. At the other end of the rocking motion, shown in the second position of the bearing 730 in FIG. 13B, the bottom of the body 736 is more distally located.

The displacement assembly 700 also includes a linkage 750 having a rigid structure configured to retain its shape when moved, such that movement can be transferred through it. In at least one embodiment, the linkage 750 may be a ball linkage having a rounded or ball-shaped end at each end of a linear bar, as in FIGS. 12-13B. However, other types of linkages 750 are also contemplated. In the embodiments of FIGS. 12-13B, each end of the linkage 750 is received and movably retained within a corresponding pocket. For instance, a first end 752 of the linkage 750 is received and retained within a pocket 738 formed in the bearing 730, such as in the distal face 737 of the bearing body 736. The opposite second end 754 of the linkage 750 is received and movably retained within a pocket 522 formed in the ablation wire mount 520. Each of the pockets 738, 522 may be formed as the socket of a ball-and-socket joint which allows rotational motion of the corresponding first or second end 752, 754 of the linkage 750 therein. However, in at least one embodiment the pockets 738, 522 may be more limited, restricting degrees of freedom of motion of the corresponding first or second end 752, 754 of the linkage 750 so movement is limited to particular directions. In the embodiment shown in FIGS. 12-13B, for instance, the pockets 738, 522 may have linear side walls which restrict lateral movement of the corresponding first or second end 752, 754 of the linkage 750 therein, but which permit movement in a vertical direction. The back wall of the pocket 738, 522 restricts movement in the axial direction. However, the pocket 738, 522 may also be sufficiently sized or configured to allow the corresponding first or second end 752, 754 of the linkage 750 to roll around therein. For instance, the pocket 738, 522 may be a sliding bead type pocket or other similar structure that permits and/or facilitates limited movement of the corresponding first or second end 752, 754 therein.

Accordingly, as the bearing 730 rocks about on an angular circular path, the first end 752 of the linkage 750 follows the movement by being movably retained within the pocket 738. The pocket 738 may be formed in any location within the body 736, such as at or near the bottom of the body 736. When the portion of the body 736 having the pocket 738 is proximally located, as in FIG. 13A, the linkage 750 is pulled proximally, and the attached ablation wire mount 520 is also pulled in the proximal direction. Accordingly, the ablation wire 230 is moved in the proximal direction. When the body 736 and pocket 738 is distally located, as in FIG. 13B, the linkage 750 is pushed distally and the attached ablation wire mount 520 is also pushed in the distal direction. Accordingly, the ablation wire 230 is moved in the distal direction. The difference between the position of the ablation wire mount 520 in the proximal and distal positions is the displacement, x, of the ablation wire 230, shown in FIG. 13B. Depending on the angle of the angled face 722 of the adaptor 720, as noted above, the displacement achieved with this displacement assembly 700 may be in the range of about 50 microns-2 mm, and more preferably in the range of about 200-750 microns.

The displacement assembly 700 may also include a guide 760 which may be affixed to the first support member 432. As shown throughout FIGS. 12-13B, the guide 760 is configured to receive and movably retain the ablation wire mount 520 therein. The guide 760 restricts the degrees of freedom of movement of the ablation wire mount 520 so that only axial movement is permitted. The ablation wire mount 520 is freely movable within the guide 760, such as slidably, along the axial direction. However, movement in other directions such as laterally, vertically or rotationally are restricted by the guide 760 surrounding the ablation wire mount 520. Accordingly, the guide 760 assists the linkage 750 in permitting movement of the ablation wire mount 520, and therefore the ablation wire 230, in the axial direction.

The handset 400 may also include a conductive lead 530 extending through the handset housing 410, such as in FIGS. 1 and 6. The conductive lead 530 may be connected to an RF source 9 exterior of the handset 400, shown in FIG. 1, and to an RF element 532 within the handset 400, shown in FIG. 6. The other end of the RF element 532 is connected to the ablation wire 230. The RF source 9 may be a standard, commercial electrosurgical power supply such as from Bovie, Erbe, Aspen, or US Endoscopy. Power will typically be supplied in a frequency range of 250 kHz to 800 kHz with either a sinusoidal or non-sinusoidal waveform. Due to the size of the probe, the power will typically be between 10 W and 50 W, usually having a sinusoidal waveform, but other waveforms and powers may also be used. When ablation is desired, the RF source 9 may be activated to generate or provide the radiofrequency (RF) energy for ablation. The conductive lead 530 conveys the RF energy into the handset 400. The connected RF element 532 routes the RF energy to the proximal end of the ablation wire 230 located within the handset 400, which is then propagated through the ablation wire 230 to its distal tip 234 and tines 242 inserted into the target tissue, such as cancerous tumor. In certain embodiments, the conductivity between distal tip 234 of the ablation wire 230 and the tines 242 may be increased by including electrically-conductive and biocompatible additives such as magnesium particles to the junction point where the tines 242 connect to the ablation wire 230. The RF source 9 may be deactivated to stop ablation.

To use the tissue ablation device 100 of the present invention, the ablation stem 200 is first inserted into the working channel 11 of a medical device 10, such as an endoscope. The sheath positioner 300 is adjusted to adjust how much of the ablation stem 200 may extend through the open end 12 of the working channel 11 when fully distally positioned, thus setting the full deployment for the ablation stem 200. The medical device 10 is then inserted into the patient and navigated, such as through the gastrointestinal tract, until the target area is reached. Navigation may be facilitated by ultrasound, echo-location, or an endoscopic camera as is customary for endoscopic procedures. Once the target tissue is reached, the sheath positioner 300 is used as described above to move the sheath 210 from a retracted position, through the open end 12 of the working channel 11 of the medical device 10 and to a deployed position in the area around the target tissue 5, such as within the intestinal tract or stomach. The cannula positioner 600 is then used as described above to move the cannula 220 from a retracted position to a deployed position, extending through the open distal end 212 of the sheath 210. During this movement, the distal end of the cannula 220 pierces the tissue adjacent to the target tissue 5, such as the intestinal wall or stomach lining, to gain access to the target tissue 5. The cannula positioner 600 may then be locked in place with the locking mechanism 650.

The ablation positioner handle 510 may then be unlocked and the repetitive vibration started. The control box 8 may be activated, or an on/off switch at the handset 400 flipped, to turn on the motor 710. The motor 710 generates repetitive oscillating vibrations that are converted to axial vibrations by the displacement assembly 700, which drives the axial movement of the ablation wire 230. The ablation positioner handle 510 is then used to move the ablation wire 230 from a retracted position to a deployed position in which the ablation wire 230 extends through the open distal end 222 of the cannula as described above and the tines 242 spread out or extend radially outwardly from the ablation wire 230. The speed of insertion from movement of the ablation positioner handle 510 affects the spread of the tines. For example, an insertion speed of about 50 mm/sec results in a tine spread of about 1.1 cm to 1.3 cm, and speeds of about 400 mm/sec result in tine spread of about 0.7 cm to 0.8 cm. These speeds are indicative of speeds used by practitioners when advancing devices through the working channel of an endoscope.

Providing repetitive axial vibrations or displacements to the tines 242 through the ablation wire 230, the tines 242 expand in a more consistent, repeatable and reliable manner that when no repetitive axial vibrations or displacements are provided. For example, in at least one embodiment, inserting the ablation wire 230 and tines 242 at 50 mm/s may result in a tine spread in the range of 0.684-1.142 cm, which is a variation of 0.229 cm. Providing repetitive axial vibration during insertion at the same speed results in tine spread in the range of 1.136-1.144 cm, which is a variation of 0.004 cm. This is a significant 98% reduction in variation by the application of repetitive axial vibration to the ablation wire 230 and tines 242. At insertion speeds of 400 mm/s, a tine spread in the range of 0.818-0.9 cm is possible, providing a variation of 0.041 cm. When repetitive axial vibration is applied, the tine spread may be altered to a range of 0.742-0.784 cm, which corresponds to a variation of 0.021 cm. This is a reduction of about 48% in variation by the application of repetitive axial vibration. These are just a few illustrative examples and are not meant to be limiting or encompassing of the insertion speeds, axial vibrations, tine spreads or variations thereof.

In addition, the repetitive vibrations allow the ablation wire 230 and tines 242 to pierce the target tissue 5, such as a tumor, despite being very thin and flexible and otherwise not able to pierce tissue. The repetitive vibrations reduce the force needed to penetrate the tissue, such as by about 50% in certain embodiments. For instance, penetration force in some embodiments may be in the range of 0.20-0.27 N without repetitive axial vibrations, which is reduced to about 0.05-0.15 N with repetitive axial vibrations. Again, these are just a few illustrative examples and are not meant to be limiting or encompassing of the possible reduction of force achievable with the present invention.

Once the distal tip 234 and tines 242 are in place in the target tissue 5, the motor 710 may be turned off, the ablation positioner handle 510 may be locked and the RF source 9 turned on. RF energy is the transmitted down the ablation wire 230 to the distal tip 234 and tines 242 to ablate the target tissue 5 as desired. When finished, the RF source 9 is turned off. In some embodiments, the heat from the RF ablation may have melted the fastener 246 holding the tines 242 to the ablation wire 230. Otherwise, the fastener 246 may be selectively removed, such as by dissolution. When the ablation positioner handle 710 is unlocked and moved to the retracted position, the end effector tines 242 remain implanted in the target tissue 5 to act as a fiducial marker for subsequent radiation treatments. The cannula 220 is then retracted with the cannula positioner 600 and the sheath 210 is retracted with the sheath positioner 300. The entire ablation stem 200 may then be removed from the working channel 11 of the medical device 10 by moving the handset 400 away from the medical device 10 and disengaging from the working channel 11 at the connector 312.

While the present invention has been described in connection with certain preferred embodiments, it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

What is claimed is:

1. A tissue ablation device, comprising:
a handset having a motor configured to generate repetitive motion, and a conductive lead configured to receive radiofrequency (RF) energy from an RF source;
a sheath having a proximal end, distal end and an opening at each of said proximal and distal ends;
an ablation wire having a proximal end, and a distal end configured to penetrate target tissue, said ablation wire disposed concentrically within and extending through said sheath, selectively and independently axially movable relative to said sheath, and selectively extendable through said opening at said distal end of said sheath to penetrate said target tissue, said proximal end of said ablation wire:
(i) connected to said motor and configured to receive and transmit said repetitive motion in an axial direction; and
(ii) connected to said conductive lead and configured to receive and transmit RF energy to said target tissue for ablation of said target tissue;
a plurality of tines each connected to said ablation wire in proximity to said distal end of said ablation wire and configured to extend radially outwardly from said ablation wire as said plurality of tines extend beyond said distal end of said sheath, said plurality of tines configured to receive:
(iii) said repetitive motion in an axial direction from said ablation wire; and
(iv) said RF energy from said ablation wire and transmit said RF energy to said target tissue for ablation of said target tissue.

2. The tissue ablation device of claim 1, wherein said sheath and said ablation wire collectively defining an ablation stem configured for insertion in and through a working channel of a medical device and is selectively extendable through a distal opening of said working channel.

3. The tissue ablation device of claim 2, wherein the medical device is an endoscope.

4. The tissue ablation device of claim 2, wherein said tines are axially aligned with said ablation wire when retained within said sheath and extend radially outwardly from said ablation wire to a three-dimensional configuration as said ablation wire is advanced from said working channel.

5. The tissue ablation device of claim 4, wherein said spherical configuration is about 0.7-1.3 cm when said ablation wire is advanced at speeds of about 50-400 mm/sec.

6. The tissue ablation device of claim 1, wherein said handset further comprises a first support member and a second support member, wherein said first and second support members are selectively and independently axially movable relative to one another and to said handset.

7. The tissue ablation device of claim 6, further comprising a sheath positioner having:
(i) a sheath extension member extending from said handset, wherein said sheath is affixed to said sheath extension member; and
(ii) a sheath positioner handle disposed adjacent to said sheath extension member and selectively movable relative to said sheath extension member to move said sheath axially between a sheath retracted position and a sheath deployed position.

8. The tissue ablation device of claim 6, further comprising an ablation wire positioner having:
(i) an ablation wire mount secured to said first support member, wherein said ablation wire is connected to said ablation wire mount; and
(ii) an ablation positioner handle connected to said first support member, accessible from outside said handset, and is selectively movable relative to said handset to axially move said ablation wire between a wire retracted position and a wire deployed position.

9. The tissue ablation device of claim 1, wherein said motor is connected to said ablation wire and configured to generate repetitive vibrations, and said ablation wire configured to transfer said repetitive vibrations to said distal tip and said plurality of tines for repetitive axial displacement.

10. The tissue ablation device of claim 9, wherein said repetitive vibrations are in the range of about 5-200 Hz.

11. The tissue ablation device of claim 9, wherein said axial displacement is in the range of about 50 microns-1.5 mm.

12. The tissue ablation device of claim 9, further comprising a displacement assembly configured to axially move said ablation wire with said repetitive vibrations by a displacement x, said displacement assembly including:
(i) said motor, wherein said motor is a rotational motor configured to generate rotational motion about an axis, said displacement assembly further configured to convert said rotational motion to axial motion;
(ii) an adaptor affixed to and rotatable with said motor, said adaptor having an extension protruding at an angle relative to said axis of said rotational motion;
(iii) a bearing having an inner ring affixed to and movable with said extension of said adaptor, an outer ring concentrically disposed about said inner ring and independently movable relative to said inner ring, a bearing body affixed to said outer ring and movable in an angular circular motion imparted from said rotational motion; and
(iv) a linkage having a first end movably received within said bearing body and an opposite second end movably received within said ablation wire mount, said linkage linearly movable with said angular circular motion of said bearing body to position said linkage between a proximal position defined by said ablation wire mount being proximally located and a distal position define by said ablation wire mount being distally located, wherein said displacement x is the distance between said proximal and distal positions.

13. The tissue ablation device of claim 1, further comprising a cannula having a proximal end, a distal end, an opening at each of said ends and a lumen extending between said ends, said cannula disposed concentrically within said sheath and selectively and independently axially movable relative to said sheath, and selectively extendable through said distal end of said sheath, and said ablation wire disposed concentrically within said lumen of said cannula.

14. The tissue ablation device of claim 13, wherein said sheath, said cannula and said ablation wire collectively define an ablation stem configured for insertion in and through a working end of a medical device and is selectively extendable through a distal opening of said working channel.

15. The tissue ablation device of claim 13, wherein said handset further comprises a proximal end, a distal end, a first support member and a second support member, wherein said first and second support members are selectively and independently axially movable relative to one another and to said handset, said tissue ablation device further comprising a cannula positioner having:
(i) a cannula extension member extending from said proximal end of said handset;
(ii) a cannula positioner handle disposed adjacent to said cannula extension member
(iii) a cannula positioner shaft extending between said cannula positioner handle and said second support member;
(iv) said proximal end of said cannula connected to said second support member;
(v) wherein said cannula positioner handle is selectively axially movable relative to said cannula extension member to axially move said cannula between a cannula retracted position and a cannula deployed position.

16. The tissue ablation device of claim 15, further comprising a collar disposed concentrically about and selectively secured to said cannula extension member, said collar having a recess formed therein configured to receive at least a portion of said cannula positioner handle therein.

17. The tissue ablation device of claim 16, wherein said collar further comprises a protrusion extending into said recess, said cannula positioner handle further comprises an axial groove extending axially from an edge of said cannula positioner handle and configured to receive said protrusion therein, a circumferential groove extending circumferentially along at least a portion of said cannula positioner handle, said circumferential groove aligned with a portion of said axial groove and configured to receive said protrusion from said axial groove when rotational motion is applied to said cannula positioner handle, said circumferential groove further configured to restrict axial movement of said protrusion in said circumferential groove.

18. The tissue ablation device of claim 1, further comprising a fastener connecting said plurality of tines to said ablation wire, wherein said fastener is selectively removable to decouple said plurality of tines from said ablation wire upon at least one of: RF energy, and application of solvent.

19. The tissue ablation device of claim 18, wherein said fastener is polyethylene glycol having a molecular weight in the range of 1,500 daltons to 40,000 daltons.

20. The tissue ablation device of claim 18, wherein said plurality of tines remain implanted in said target tissue following removal of said fastener and retraction of said tissue ablation device from said target tissue.
